(19) United States
(12) Patent Application Publication
(10) Pub. No.: US 2014/0046640 A1
(43) Pub. Date: Feb. 13, 2014

(54) Telenostics for medical uses

(71) Applicant: Ronald E. Wagner, Fleming Island, FL (US)

(72) Inventor: Ronald E. Wagner, Fleming Island, FL (US)

(73) Assignee: BAE Systems Information and Electronic Systems Integration, Inc.

(21) Appl. No.: 12/807,925

(22) Filed: Sep. 16, 2010

Related U.S. Application Data
(60) Provisional application No. 61/342,131, filed on Apr. 9, 2010.

(51) Int. Cl. G06G 7/60; G01N 33/48; G06F 17/18; G06F 19/00

(52) U.S. Cl. 703/11; 702/19; 702/181

(57) ABSTRACT

Telenostics style prognostication algorithms are used in medical prognosis to predict the mean time to an unacceptable result, such as death, or the mean time to a cure or partial cure.

### RELATED APPLICATIONS

This Application claims rights under 35 USC §119(e) from U.S. application Ser. No. 61/342,131 filed Apr. 9, 2010, the contents of which are incorporated herein by reference.

### FIELD OF THE INVENTION

This invention relates to health maintenance and more particularly to the utilization of a telenostics-style prognostication algorithm for use in medical prognosis to predict the mean time to an unacceptable result or the mean time to cure or partial cure.

### BACKGROUND OF THE INVENTION

As discussed in U.S. patent application Ser. No. 12/548,683 by Carolyn Spier filed on Aug. 27, 2009, assigned to the assignee hereof and incorporated herein by reference, prognostication algorithms including reasoners and the like are created using modeling and simulation to define a performance vector for creating a prognostication algorithm. The prognostication algorithm when created then takes sensor or monitored results and makes a prediction of the probability of a failure mode so that for instance vehicle maintenance organizations can proactively react to the prediction of a failure mode and provide remedial action.

Up until the present time, this type of prognostication algorithm has not been utilized in the medical field.

As discussed in U.S. Pat. No. 5,769,074 issued to Stephen Barnhill and Zhen Zhang, the inventors use support vector machines to find a fit performance window. However, in their systems they require an entire field of data, such as that provided for instance by taking the DNA of anybody from whom they can collect a specimen. The system then tries to match the patient's specific DNA to that field and then calculates the probability of that DNA occurring within the entire database. To say the least, this is a daunting task and Barnhill and Zhang utilize support vector machines to help them narrow down the data and find a curve fit for a specific string of DNA throughout the entire field.

The problem is that in order to correctly calculate probability, one has to access an enormously large database and with this amount of data very powerful computers, including cluster computing, are required to enable vector machine operation, for instance to find a fit for a specific string of DNA against a huge database.

While support vector machines have been utilized to do DNA matches, when applied to the medical arena, one has essentially two problems. The first problem is how one builds an exceedingly large database. The second problem is that even with a support vector machine, one has the problem of the length and difficulty of the processing to match up a specific DNA strain to the DNA strings of a huge population.

This support vector machine application essentially is one of matching to see if a particular disease exists or is likely to exist. Because of the difficulties associated with support vector machines, another modality is required in order to ascertain the probability that a disease exists, and of a high probability, the mean time to failure of a body structure or organ as a result of this disease. Failure in this case means an unacceptable result, including for instance morbidity.

Thus, in the past there have been no attempts to automate prognosis, much less to render prognosis in terms of mean time to failure or mean time to an unacceptable result, or for that matter mean time to a successful treatment or cure.

### SUMMARY OF INVENTION

In order to narrow down the data processing requirements and to provide a different type of attack on the problem of prognostication, in one embodiment patient monitoring provides a data set which suggests a particular algorithm for characterizing a particular disease. This data set is utilized in a modeling and simulation step to define a performance vector through the data that is used to establish the particular prognostication algorithm for the particular patient.

In one embodiment the data available from the patient is utilized to define an initial prognostication algorithm which then may be refined by generating a synthetic data set based on nearly orthogonal latin hypercubes. This synthetic data set is then used to alter the coefficients of the prognostication algorithm to produce an algorithm which produces more highly accurate prognostications or predictions. In short, the subject system is an automatic prognosis generating system.

In order to attack the problem of being able to predict the outcome of treatments or the course of a disease, the subject system treats the human body as a system having failure modes. These failure modes are predicted using the PRDICTR type algorithms described in U.S. patent application Ser. No. 12/548,683 by Carolyn Spier filed on Aug. 27, 2009, assigned to the assignee hereof and incorporated herein by reference. To predict the failure modes in a human system, one must first collect data for a given disease, for example diabetes, and then create data collection methods that are specific to the disease, in this case diabetes.

By so doing, one is essentially narrowing the performance window and in order to obtain a general population probability of having the specific disease, rather than collecting data on every DNA strain of everybody in the world, it is only necessary to collect data for those people in the population that have the probability of the particular disease. One then creates a personal history. Once one has a personal history one can use the subject technique to calculate the probability of a particular disease course, meaning predicting mean time to failure, however defined, or mean time to success, however defined.

In the subject system the PRDICTR algorithms are applied to the human body which is after all a system. In these techniques the PRDICTR system starts with a top down functional breakdown of the performance characteristics of the system. For instance, structural characteristics would include bones, whereas functional characteristics which would include organs. Analysis is then performed as to the cost significance and maintenance significance of the particular characteristics.

There are categories of the human system which the top down functional breakdown identifies. These categories identify the failure modes. For instance, a failure mode might be heart failure, or might be the failure of a bone.

These failure modes can then be used to create a criticality analysis of the impact of a failure mode, for instance the impact of a broken bone or the impact of heart failure, which then identifies what further data is necessary to be collected.

Thus, rather than the support vector machine which tries to find a match between existing data and a large population base, with the subject invention one narrows down the database to look for specific data that is collectable and definable against the probability of a specific disease.

Once one has identified the probability of a failure mode (particular disease) that one wants to monitor, one can then begin to add other factors that would be associated with that failure mode, with the other factors becoming a vector to create a refined prognostication algorithm associated with a specific patient. For instance the patient could be a 50 year old who flies airplanes upside down and has a mother who has diabetes.

It will be appreciated that this algorithm is very narrow and is specific to a particular person which results in the ability to accurately predict the probability of a specific disease.

Note that by limiting the type of data, it may be that the sample size is relatively small.

As discussed in Provisional Patent Application No. 61/342,100, filed Apr. 9, 2010 entitled Nearly Orthogonal Latin Hypercubes for Optimization Algorithms, in order to increase the sample data set to ensure accurate predictions, one initially creates a prognostication algorithm from a small select data window and then augments the sample size or enhances it utilizing for instance nearly orthogonal latin hypercube techniques to be able to improve the coefficients of fit in the prognostication algorithm. This is done through the nearly orthogonal latin hypercube technique to create synthetic data.

By utilizing the synthetic data one generates an improved prognostication algorithm based on a specific vector or probability of a failure mode and then turns that specific vector into an improved probability estimate that the patient would have a particular disease or that an undesired result is going to occur at some specific time in the future.

Having then limited the data and provided improved performance vectors, one can use the modeling and simulation tools described in the above referenced patent application to evaluate the contribution of certain factors over others. Age versus occupation versus medical history could be evaluated and one could obtain a performance algorithm that would calculate the probability of a disease taking place or the probability of an undesired result.

If one followed the above procedures one could narrow the data that one needs to collect on a person to a set of factors that have the highest contribution to that performance vector or that fault mode, i.e. undesired result. One would then be able to monitor the factors that were used in determining the particular disease involved and to prescribe a treatment aimed to increase the mean time to an unacceptable result or decrease the mean time to effectuate a cure.

In summary, if one utilizes prognostication techniques in a top down modeling and simulation scenario to characterize the human body as a system, one can define human failure modes and successfully establish a prognosis and a time line.

Thus, telenostics style prognostication algorithms are used in medical prognosis to predict the mean time to an unacceptable result, such as death, or the mean time to a cure or partial cure.

### BRIEF DESCRIPTION OF THE DRAWINGS

These and other features of the subject invention will be better understood in connection with the Detailed Description, in connection with the Sole Drawing FIGURE, of which:

The Sole Drawing FIGURE is a diagrammatic illustration of the utilization of prognostication in medical prognosis in order to provide the probability of either a disease being present, or having ascertained a probable disease, the mean time to an unacceptable result, or the mean time to effectuate a cure.

### DETAILED DESCRIPTION

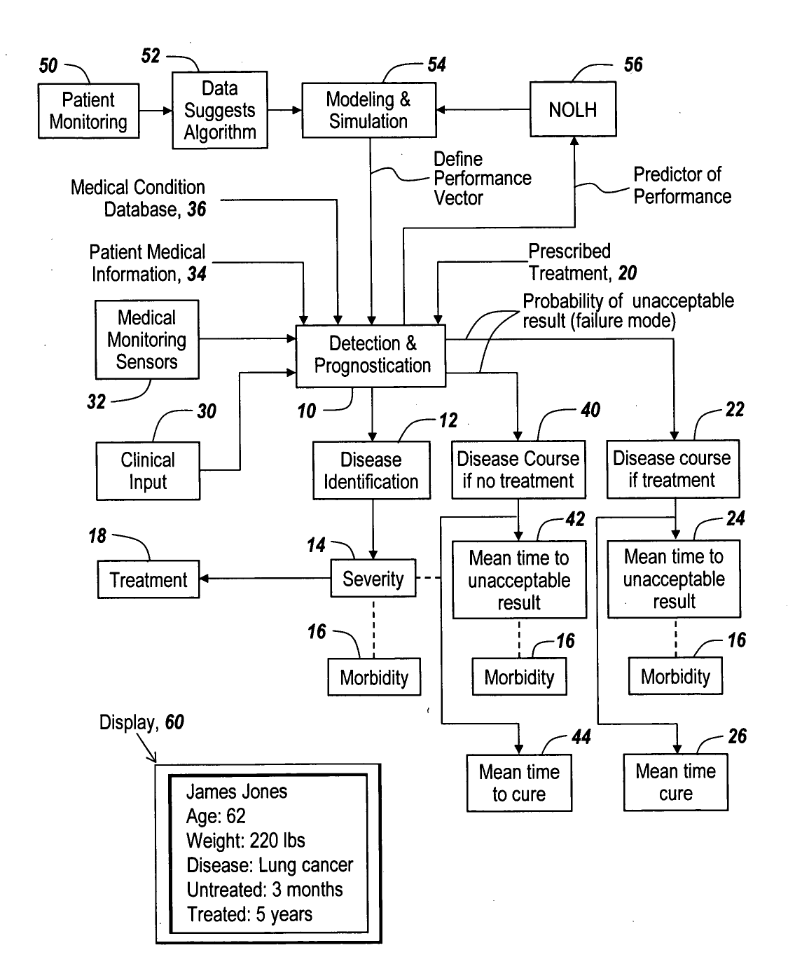

Referring now to FIG. 1, in the subject system a detection and prognostication set of algorithms 10 is utilized to provide detection and prediction of a specific disease in terms of disease identification 12, the severity thereof 14 and potential morbidity of the patient as a result of this disease as illustrated at 16. Having identified the disease at its severity, treatment options 18 may be formulated.

Having identified the probability that a particular disease is present, assuming that prescribed treatment 20 is applied to the patient, then the detection and prognostication algorithms 10 predict the disease course if treatment is applied as illustrated at 22 which results in either mean time to an unacceptable result 24, or what happens if one fails to treat the disease properly. In either case, the result may be morbidity 16. Alternatively, the mean time to cure as illustrated at 26 is calculated, again based on the probability produced by the detection and prognostication algorithms such as those described in the aforementioned US patent application involving the so-called PRDICTR algorithms.

It is noted that a prediction algorithm operates on a defined performance vector. This performance vector takes into account clinical input 30, an input from medical monitoring 32, as well as patient medical information 34, and also data from a medical condition database 36 to arrive at the probability of an unacceptable result or a cure. This is expressed in terms of mean-time-to-failure and mean-time-to-cure. In the illustrated example the probability is based on actually treating the disease.

The same type of probability of unacceptable result or mean time to cure can be computed for the disease course if no treatment as illustrated at 40. This results in the mean time to an unacceptable result 42 including morbidity 16; or the mean time to a cure 44.

Thus as part of the subject invention it is the application of prognostication techniques utilizing the top down modeling and simulation for defining a performance vector that leads to a robust algorithm for predicting mean-time-to-failure for a system, where in the system is the human body, and where failure relates to the failures of organs, structures, or functions of the human body.

In one embodiment, in order to define the performance vector used to create prognostication algorithms 10, one utilizes a data set derived from patient monitoring 50 to provide a data set 52 that suggests the particular algorithm to be used for the particular patient and his particular disease.

This data set is then modeled and simulated as illustrated at 54 to define a performance vector that is utilized in prognostication algorithm 10.

It is part of the subject invention to refine the performance vector through adjustment of the modeling and simulation coefficients by operating on an expanded data set which takes the initial algorithm 10 and synthetically produces data, for instance by utilizing a nearly orthogonal latin hypercube methodology 56, to generate synthetic data. This synthetic data set is then used to refine the coefficients in the prognostication algorithm.

The result is a display 60 of the identity of the patient his age weight and disease, along with a prognostication of his lifespan either in the untreated case or the treated case.

Display 60 may also be used to display a wide variety of predictable outcomes given the probability associated with the performance vector defined through the modeling and simulation, as well as the ongoing collection of data which refines the probability of a particular disease and/or the effects of the particular disease on the system, namely the human body.

In summary, what has not been heretofore been available is a systematic mathematically-derived prognostication technique for evaluating disease and the course of the disease so as to be able to output the mean-time-to-failure of a failure mode, wherein the failure mode is a failure of an organ, structure, or function of the human body.

While the present invention has been described in connection with the preferred embodiments of the various figures, it is to be understood that other similar embodiments may be used or modifications or additions may be made to the described embodiment for performing the same function of the present invention without deviating therefrom. Therefore, the present invention should not be limited to any single embodiment, but rather construed in breadth and scope in accordance with the recitation of the appended claims.

1. An apparatus for providing recommendations in treatment of a human body, comprising:
a processor;
a computer readable storage medium encoded with instructions which when executed by the processor, performs:
suggesting an algorithm that characterizes a particular disease based on data obtained from patient monitoring information, the suggested algorithm being used in a modeling and simulation step to define a performance vector for creating a prognostication algorithm;
determining a probability of the particular disease is prevalent, and having identified the probability of the particular disease, determining a probability of mean time to an unacceptable result or mean time to an acceptable result by operating the prognostication algorithm on clinical data, data from medical monitoring and the patient monitoring information, and data from a medical condition database, wherein the unacceptable result comprises treatment failure and the acceptable result comprises a cure or partial cure; and
providing recommendations of a treatment regimen including the probability of the mean time to the unacceptable result or the mean time to the acceptable result based on the identified disease; and
a display for providing for outputting said recommendations.

2. (canceled)

3. The apparatus of claim 1, wherein the prognostication algorithm includes a detection algorithm.

4. The apparatus of claim 1, wherein the prognostication algorithm determines a severity of the disease.

5. The apparatus of claim 4, wherein the processor recommends the treatment regimen based on the disease identification and the severity of the disease.

6. The apparatus of claim 5, wherein the prognostication algorithm determines the probability of disease course if left untreated based on the severity of the disease.

7. The apparatus of claim 6, wherein the mean time to the acceptable result is based on untreated disease course.

8. The apparatus of claim 7, wherein the prognostication algorithm determines the probability of the mean time to cure if the disease is left untreated, the cure depends on the body's response to the identified disease.

9. The apparatus of claim 4, wherein the disease identification along with the severity and treatment defines a predicted disease course.

10. The apparatus of claim 9, wherein the predicted disease course is used by the prognostication algorithm to calculate the mean time to the unaccepted result.

11. The apparatus of claim 9, wherein the predicted disease course is used by the prognostication algorithm to calculate the mean time to cure or partial cure.

12. (canceled)

13. The apparatus of claim 1, wherein the unacceptable result includes morbidity.

14. (canceled)

15. (canceled)

16. The apparatus of claim 1, wherein the prognostication algorithm is derived utilizing the modeling and simulation steps and wherein the modeling and simulation step defines the performance vector through the data, the defined performance vector being utilized in the prognostication algorithm.

17. The apparatus of claim 1, wherein an output of the prognostication algorithm is a predictor of performance.

18. The apparatus of claim 17, wherein the predictor of performance is used by a nearly orthogonal latin hypercube process to expand the data used in the modeling and simulation steps.

19. The apparatus of claim 1, and further including displaying a name of the patient, patient parameters, identity of the disease, and prognosis if the disease is left untreated and prognosis if the disease is treated.
